(19) United States
(12) Patent Application Publication
(10) Pub. No.: US 2003/0124152 A1
(43) Pub. Date: Jul. 3, 2003

(54) Use of decorin in a cosmetic or dermatologic composition

(76) Inventor: Danny Zhong Der Pang, Fullerton, CA (US)

Correspondence Address: Danny Z. Pang, 1526 W. Flower Avenue, Fullerton, CA, 92833, US

(21) Appl. No.: 10/004,176

(22) Filed: Nov. 2, 2001

(51) Int. Cl. A61K 38/48; A61K 7/00

(52) U.S. Cl. 424/401; 424/94.63

(57) ABSTRACT

The present invention relates to the use of decorin for the preparation of a cosmetic or pharmaceutical or dermatological composition for treatment of intrinsic (due to genetic factors) and extrinsic (due to the environmental factors) aging of the skin. Decorin naturally exists in the skin to regulate skin collagen fiber formation, prohibit collagen fibrillogenesis, prevent collagen degradation, enhance collagen stability, and normalize skin cells and elastin fibers. The composition can effectively treat and prevent skin aging, repair damaged skin from aging and restore skin to a more resiliency and youthful appearance. The present invention also includes methods of applying the compositions to the skin.

### BACKGROUND OF THE INVENTION

The present invention relates to the use of decorin for the preparation of a cosmetic or pharmaceufical or dermatological composition for effectively treating and preventing intrinsic (due to genetic factors) and extrinsic (due to environmental factors) aging of the skin and methods for treatment and prevention of skin aging using such compositions.

Human Skin

The skin is composed of two mutually dependent layers: the outer epidermis and inner dermis, both cushioned on the fat-containing subcutaneous tissue, the panniculus adiposus. The stratified cellular epidermis contains two main zones of cells (keratinocytes), an outer layer of anucleate cells known as the stratum corneum, and an inner region of viable cells, the stratum germinativum. There are three strata of cells in the germinativum, basal, spinous, and granular layers, each representing a progressive stage of differentiation and keratinization of the epidermal cells as they evolve into the dead, tightly packed stratum corneum cells on the skin surface. The stratum corneum layer is the stiffest layer of the skin, preventing water loss from the inner epidermal hydrated layers and shielding against damage from the environment.

Dermis makes up 90% of the thickness of the skin and consists of a three-dimensional extracellular matrix (ECM) of loose connective tissue composed of highly stable fibers of collagen and elastin. These fibrous proteins are embeded in amorphous ground substances such as glycosaminoglycans, decorin, biglycan, and osteopontin. Collagen has a high tensile strength to prevent skin from being torn by over stretching. Collagen molecules resemble over one another oriented somewhat parallel to the epidermis, giving skin its softness and resiliency. Being particularly abundant over the face and neck, coarse elastic fibers are entwined in the collagenous fibers, allowing its movement.

The skin provides protective functions of importance to our survival. These functions can be detrimentally affected by the changes in the skin structure due to aging.

Deformable Lipidvesicles

Normal skin is impermeable even for low molecular weight (>500 dalton) agents due to the excellent barrier of the stratum corneum, whose maximum pore size (virtual pores) is only around 20-30 nm (Paul et al., Vaccine 16:188-95, 1998). However, deformable lipidvesicles, such as liposomes, transfersomes, or ethosomes, are able to easily penetrate the surface skin to underlying layers. (Cevc, G. Crit. Rev. Ther. Drug Carrier Syst. 13:257, 1996; Hoffer et al., Anticancer Res. 19:1505, 1999; Cevc et al., Biochim. Biophys. Acta 1368:201, 1998; Barry et al., Eur. J Pharm. Sci. 14:101, 2001). Lipidvesicles, made of a variety of fatty substances, such as phospholipids and phosphatidylcholine, form microscopic sacs or spheres, when properly mixed with water or ethanol. Lipidvesicles can trap any substance that will dissolve in water or oil. Different lipidvesicles possess a different magnitude of deformability and skin penetration. For example, transfersomes, consisting of natural phosphatidylcholine and sodium cholate, have a magnitude of deformability several orders higher than liposomes and are able to spontaneously pass through the narrow pores of the skin, which are 4 to 10 fold smaller than themselves (Cevc et al., Biochim. Biophys. Acta 1368:201, 1998; Cevc et al., Adv. Drug Deliv. Systems 18:349, 1996). This phenomena of the skin penetration has been explained when transfersomes are applied to the intact non-occluded skin, due to dehydration by evaporation, they create an osmotic pressure difference between the region of higher water concentration inside the skin and the nearly dry surface of the skin. To avoid the osmotic tension by dehydration, the transfersomes open narrow intercellular pores of the skin and penetrate through the barrier (Hofer et al., Anticancer Res. 19:1505, 1999; Cevc et al., Biochim. Biophys. Acta 1104:226, 1992).

There have been many successes in the applications of the lipidvesicles as a drug carrier for transporting a large polypeptide such as IFN-alpha, IL-2 or insulin through the normal intact skin into the body (Paul et al., Vaccine Res. 4:145, 1995; Paul et al., Eur. J Immunol. 25:3521, 1995; Cevc et al., Biochim. Biophys. Acta 1368:201, 1998; Hofer et al., Anticancer Res. 19:1505, 1999; Barry et al., Eur. J Pharm Sci. 14:101, 2001; Touitou et al., J. Control Release 65:403, 2000). As a delivery system, deformable lipidvesicles have been used cosmetically or dermatologically to deliver large active anti-aging molecules, such as proteins or peptides, into the skin for combating skin aging (Weiner et al., J. Drug Target. 2:405, 1994; Cevc, G. Crit. Rev. Ther. Drug Carrier Syst. 13:257, 1996; Perugini et al., Int. J Pharm. 196:51, 2000; Fiume, Z. Int. J Toxicol. 20 Suppl 11:21, 2001).

Collagen

Collagen, synthesized by fibroblasts, represents a large family of glycoproteins which are located in the extracellular matrix. 20 different types of collagens (types I-XX) have been defined so far and can be divided into two major groups, i.e., fibrillar and non-fibrillar collagens. Types I, II, III, V, and XI constitute the fibrillar collagens, whereas types IV, VI to X, and XII to XX represent the structurally diverse, non-fibrillar members (Myers et al., Am. J. Pathol. 151:1729, 1997; Koch et al., J. Biol. Chem. 276:23120, 2001). They all share a triple-helical segment of variable length (100-450 nm) but differ considerably in the size and nature of their globular domains. They play different functions including the formation of fibrils, microfibrils and networks. For example, collagens types I, II, III, V, and XI participate in quarter-staggered fibrils, while types IV, VIII, IX, and VI form beaded filaments or membrane-associated sheets. Types XII, XIV, IX and XX are fibril-associated with interrupted triple helices (FACITs) to connect fibrils to other matrix elements, and type VII forms anchoring fibrils. Type X can only be found in matrix of the hypertrophic zone of the epiphyseal growth plate cartilage and is probably involved in the process of mineralization and endochondral ossification. Type XVIII collagen containing both a frizzled and an endostatin domain involves coordination between inductive signals and matrix molecules (Martin et al., Trends Biochem. Sci. 10:285, 1985; van der Rest and Garrone, FASEB J 5:2814, 1991; Brown and Timpl, Int. Arch. Allergy Immunol. 107:484, 1995; Pihlajaniemi and Rehn, Prog. Nucleic Acid Res. Mol. Biol. 50:225, 1995; Sutmuller et al., Histol. Histopathol. 12:557, 1997; Lin et al., Development 128:1573, 2001; Koch et al., J. Biol. Chem. 276:23120, 2001).

Collagen is the major constituent of skin and constitutes more than 70% of the mass of the skin in terms of its dry weight. Among the collagens of the skin, fibrillar collagens type I and II are predominate, about 90% type I and 10% type II (Epstein and Munderloh, J. Biol. Chem. 253:1336, 1978; Fukar et al., Acta Derm. Venereol. 68:196, 1988; Clore et al., Biochim. Biophys. Acta 586:384, 1979; Chan and Cole, Anal. Biochem. 139:322, 1984). Normally, collagen molecules stay soluble and slide over one another, giving skin its softness, strength, and resiliency. When collagen molecules are damaged by cross-linking, they become stiff and inflexible, lossing their elasticity.

Decorin

Decorin belongs to a family of small leucine-rich dermatan sulfate proteoglycans, including biglycan, fibromodulin, lumican, epiphycan, and katakana, which are similar in their general structures but genetically differ (Iozzo and Murdoch, FASEB J 10:598, 1996). Decorin is an ubiquitous component of the ECM and plays several important functions in cell growth and development and maintenance of the ECM structure including collagen fibril assembly and stability (Scholzen et al., J. Bio. Chem. 269:28270, 1994; Sanatra et al., Clin. Invest. 100:149, 1997; Santra et al., Proc. Natl. Acad. Sci. USA 92:7016, 1995; Neame et al., Cell Mol. Life Sci. 57:859, 2000; U.S. Pat. No. 6,046,162).

Decorin, also known as PG-II, PG-40 or PG-S2, or Dcn, consists of a 40-kDa core protein, a single glycosaminoglycan (GAG) chain of the chondroitin/dermatan sulfate type, and three N-linked oligosaccharides located on the same side of the molecule (Pearson et al., J. Biol. Chem. 258:15101, 1983; Day et al., Nucleic Acids Res. 14:9861, 1986; Iozzo and Murdoch, FASEB J. 10:598, 1996; Ramamurthy et al., J. Biol. Chem. 271:19578, 1996; Gu et al., Bioch. biophys. Res. Commun. 232:91, 1997). The decorin core protein contains a central region comprising 8-12 homologous leucine-rich repeat units. Each unit contains 21-29 amino acid residues and a characteristic alpha-helix/beta-sheet folding pattern (Day et al., Nucleic Acids Res. 14: 9861, 1986; Kobe and Deisenhofer, Nature 366:751, 1993; Iozzo R., Crit. Rev. Biochem. Mol. Biol. 52:141, 1997). The decorin core protein has been sequenced (Krusius and Ruoslahti, Proc. Natl. Acad. Sci. USA 83:7683, 1986; Day et al., Biochem. J. 248:801, 1987), and its gene has also been localized on human chromosome 12 (McBride et al., Genomics. 6:219, 1990). The GAGs are unbranched polysaccharides containing repeating disaccharide units, highly sulfated and highly negatively charged (Krusius and Ruoslahti, Proc. Natl. Acad. Sci. USA 83:7683, 1986).

Decorin binds to certain types of collagens, such as type I, II, IV, VI, and XIV and delays in vitro fibrillogenesis (Vogel et al., Biochem. J. 223:587, 1984; Schmidt et al., J. Cell Biol. 104:1683, 1987; Pogany & Vogel, Biochem. Biophys. Res. Commun. 189:165, 1992; Bidanset et al., J. Biol. Chem. 267:5250, 1992; Schonherr et al., J. Biol. Chem. 270:8877, 1995; Keene et al., J. Biol. Chem. 275:21801, 2000; Bidanset et al., J. Biol. Chem. 267:5250, 1992; Wiberg et al., J. Biol. Chem. 276:18947, 2001; Ehnis et al., J. Biol. Chem. 272:20414, 1997). Decorin binds to transforming growth factor-beta (TGF-beta) to affect cell proliferation and reduce or inhibit wound contraction (Border et al., Nature 360:361, 1992; Isaka et al., Nature Med. 2:418, 1996; U.S. Pat. Nos. 6,046,162, 6,277,812 and 5,583,103). Studies indicated that transgenic mice that overexpress a dominant negative-type II TGF-beta receptor (delta beta RII) in the epidermis exhibites a thickened and wrinkled skin. And histologically the epidermis was markedly hyperplastic and hyperkeratotic (Wang et al., Proc. Natl. Acad. Sci. USA 94:2386, 1997). Binding of decorin to TGF-beta can block the stimulation TGF-beta receptor that is required to mediate the epidermal effects of TGF-beta on the skin. Decorin also binds to fibronectin, C1q, receptors of epidermal growth factor (EGF) and endocytosic membrane receptors (Schmidt et al., J. Cell Biol. 104;1683, 1987; Krumdieck et al., J. Immunol. 149:3695, 1992; Yamaguchi et al., Nature 346:281, 1990; Gloss et al., Biochem. J. 215:295, 1983; Renato et al., J. Biol. Chem. 274:4489, 1999; U.S. Pat. No. 6,277,812). Decorin binding to fibrillar collagens or several other components of the extracellular matrix is predominately involved in its core protein (Pogany & Vogel, Biochem. Biophys. Res. Commun. 189:165, 1992; Schonherr et al., J. Biol. Chem. 270:8877, 1995; Ehnis et al., J. Biol. Chem. 272:20414, 1997; Keene et al., J. Biol. Chem. 275:21801, 2000), although decorin interaction with type XIV collagen is mediated through its glycosaminoglycan side chain (Font et al., J. Biol. Chem. 268:25015, 1993; Ehnis et al., J. Biol. Chem. 272:20414, 1997).

Decorin is synthesized by fibroblasts and can be isolated from many different tissues, including skin, cartilage, and tendon (Choi et al., J. Biol. Chem. 264:2876, 1989; Rosenberg et al., J. Biol. Chem. 260:6304, 1985). Many factors, in particular, aging, influences the production of decorin. Senescent fibroblasts lose their ability to synthesize decorin. Aging affects not only the level of decorin synthsis and the size and polydispersity of the core proteins of decorin, but also the synthesis of glycosaminoglycan of decorin (McAlinden et al., Osteoarthritis Cartilage 9:33, 2001; Carrino et al., Arch. Biochem. Biophys. 373:91, 2000; Takeda et al., J. Cell Physiol. 153:450, 1992; Schonherr et al., Biochem J. 290:893, 1993; Vural et al., Mech. Ageing Dev. 77:97, 1994; Fedarko et al., J. Cell Physiol. 151:215, 1992; Brown et al., Mech. Ageing Dev. 77:97, 1994; Biecker and Schachtschabel, Z. Gerontol. Geriatr. 32:124, 1999). Chronic sun exposure or photodamaged skin significantly reduces the synthesis of decorin (Bernstein et al., Lab. Invest. 72:662, 1995). Some patients with Marfan's or Ehlers-Danlos syndrome shows the deficiency of the decorin core protein (Wu et al., J. Dermatol. Sci. 27:95, 2001; Beavan et al., J. Biol. Chem. 268:9856, 1993). Deficiency of decorin is directly linked to a totally disorganized extracellular matrix which lacks shape modules or interfibrillar proteoglycan bridges (Scott et al., Exp. Cell Res. 243:59, 1998). Deficiency of decorin also affects the morphology of skin fibroblasts (Gu and Wada, J. Biochem. (Tokyo) 119:743, 1996) and causes abnormal morphology of collagen fibril in skin and skin fragility with markedly reduced tensile strength (Danielson et al., J. Cell Biol. 136:729, 1997; Vogel and Trotter, Collagen Relat. Res., 7:105, 1987).

Since the level reduction of skin decorin is accordingly associated with a decrease in the tensile strength of the skin causing wrinkles and laxity, to boost the synthesis of decorin in the skin by topical use of conjugated linoleum acid, petroselinic acid, and/or derivatives, can improve the condition and appearance of the skin (U.S. Pat. Nos. 6,042,841 and 6,287,553).

Decorin has also been produced in vitro by a mean of recombinant technology in E. coli, mammalian cells or insect cells (Ramamurthy et al., J. Biol. Chem. 271:19578, 1996; Hering et al., Anal. Biochem. 240:98, 1996; Gu et al., Biochem. biophys. Res. Commun, 232:91, 1997).

Skin aging

Cutaneous aging is a complex biological phenomenon consisting of two distinct components, (a) the intrinsic process of senescence or genetically determined degenerative aging processes and (b) the extrinsic damage induced by chronic exposure to UV radiation, chemicals, cigarette smoking or air pollution. These two processes are superimposed in the sun-exposed areas of skin, with profound effects on collagen and the elastic fiber network of the skin, causing the destruction of skin architecture and the dramatic alterations in the appearance of the integument with increasing age.

Intrinsic aging is largely based on (genetics and/or hormones) hereditary factors. A substantial amount of circumstantial evidence indicates that senescence affects the synthesis and degradation of skin collagen, apoptosis, and metalloproteinase expression and its proteolytic activity. As skin ages, fibroblasts lose their ability to react to growth factors for the proliferation and synthesis of collagen and the dermis and the epidermis become thin (West, Arch. Dermatol. 130:87, 1994). Examination by scanning electron microscopy revealed a decrease in the number of collagen fibre bundles in normal human skin with age (Lovell et al., Br. J. Dermatol. 117:419, 1987). The synthesis of collagen type I in the eyelid skin is diminished with age (DeBacker et al., Ophthal. Plast. Reconstr. Surg. 1413, 1998). It has been found that the decrease collagen synthesis is in a statistically significant linear manner with age (Dumas et al., C. R. Acad. Sci. 319:1127, 1996; Phillips et al., J. Invest. Dermatol. 103, 228, 1994; Uitto and Bernstein, J. Investig Dermatol. Symp. Proc. 3:41, 1998). Efficiency of apoptosis is decreased during aging and may also contribute to the alterations of skin aging (Haake et al., J. Investig Dermatol. Symp. Proc. 3:28, 1998). In addition, overexpression of matrix metalloproteinase (MMP) collagenase, 92 kDa gelatinase, and stromelysin in the senescent dermal fibroblasts may explain the age-related atrophy of extracellular matrix architecture. MMP is considered a major enzyme involved in the degradation of matrix components, including collagen. It has been indicated that both collagenous and elastic components display a degeneration consistent with the overexpression of proteolytic activity. Skin collagen changes its solubility, becomes cross-linking, and loses its elasticity. Elastin undergoes irreversible structural and compositional changes, such as deposition of osmiophilic materials or substitution of the amorphous elastin with interwoven filaments (Pasquali-Ronchetti and Baccarani-Contri, Microsc. Res. Tech. 38:428-35, 1997; West, Arch. Dermatol. 130:87, 1994; Nusgens et al., J. Invest. Dermatol. 116:853, 2001; Burke et al., Exp. Gerontol. 29:37, 1994).

Extrinsic aging of the skin has also long been recognized and is directly associated with enviromental factors, such as, ultraviolet (UV) radiations from sunlight, chemicals, cigarette smoking, and air pollution. There is a cause-and-effect relationship between prolonged and/or repeated exposure to UV radiation and the premature aging of skin, known as photoaging. Excessive exposure to UV radiation contributes substantially to premature reduction in the quality and quantity of collagen in the skin. Collagen becomes cross-linking and lose its solubility and elasticity by UV irradation (Bailey, A. Mech. Ageing Dev. 122:735, 2001). Collagen synthesis is reduced in photoaged skin by approximately 45% compared to protected skin (Kligman et al., Photodermatol. Photoimmunol. Photomed. 16:224, 2000). UV irradiation induces overexpression of matrix metalloproteinases (MMP) in skin to accelerate the disappearance of collagen contents in the photoaged skin (Fisher et al, J. Invest. Dermatol. 117:219, 2001). UV irradiation acts in an additive manner with tobacco smoke to further speed premature aging of human skin. It appears that smokers look older than non-smokers of the same age, for smoking can induce MMP, which might be important in the skin-ageing effects of tobacco smoking (Yin et al., Photodermatol. Photoimmunol. Photomed. 17:178, 2001; Lahmann et al., Lancet 357:935, 2001; Yin et al., J. Dermatol. Sci. Suppl 1:26, 2001). Studies revealed that the induction of MMP by UV irradiation is a consequence of activator protein (AP)-1 and nuclear factor (NF)-KB activation as well as mutations of mitochondrial DNA (Berneburg et al., Photodermatol. Photoimmunol. Photomed 16:239, 2000).

Damage to the skin by UV radiation includes irreversible alterations in the composition, organization, and structure of the collagenous extracellular matrix in the dermis. It is believed that the breakdown of dermal interstitial collagen is followed by imperfect repairing or imperfect remodeling. The imperfect remodeling, however, further alters the structural integrity of the dermis. The repeated cycles of the collagen degradation and imperfect remodeling result in accumulation of the amorphous elastin with interwoven filaments in the superficial dermis, showing an old and wrinkled appearance. Hence, photoaging can be characterized histologically by diminution of collagen, and ultrastructural alterations of collagen fibrils, such as cross-linking or chain cleavage, and accumulation of elastotic material in the papillary dermis. Clinically, photoageing is characterized by coarseness, wrinkling, mottled pigmentation, laxity, telangiectasia, lentigenes, and benign as well as malignant neoplasms.

Both aging processes, intrinsically and extrinsically, act synergistically to alter the structure, organization, and composition of the two major structural proteins of the skin, collagen and elastin. These changes manifest themselves externally by signs of aging, such as lines, wrinkles, loss of elasticity, sagging, skin dryness and unevenness, blotches, age spots, pigmented spots, and benign and malignant neoplasms.

To counteract the undesired effects of both types of skin aging (i.e. intrinsic and extrinsic), there is a steadily increasing effort to use cosmetic and/or dermatological compositions containing active agents for treating or reparing or delaying the visible signs of skin aging. Unfortunately, these anti-aging active agents suffer from the major drawback of causing skin allergy reactions, discomforts or other side effects after their application, some agents can even make people more susceptible to the damaging effects of the sun, accelerating photoaging of the skin (Information from the Center for Food Safety and Applied Nutrition, Cosmetic, FDA, USA Mar. 7, 2000; Held et al., Contact Dermatitis 40:310, 1999; Perrenoud et al., Dermatology 189:225, 1994; Augustin et al., Skin Pharmacol. 10: 63, 1997; Kim et al., J. Inv. Dermat. 98:359, 1992; Griffiths et al., N. Eng. J. Med. 329:530, 1993; Chojkier et al., J Biol. Chem. 264:16957, 1989; U.S. Pat. Nos. 5,747,538, 5,747,049, 5,386,012 and 6,203,805).

Thus, there continues to be a need for effective cosmetic/dermatological compositions for topical application to skin for treating, reparing and delaying the visible aging signs of skin including wrinkles, lines, sagging, age spots, loss of elasticity, or hyperpigmentation. There also remains a need for compositions for effectively treating and preventing skin aging which do not possess those drawbacks when applied to the skin. There also remains a demand for anti-aging agents which are able to modulate skin collagen network, maintain and restore the solubility and elasticity of skin collagen, prevent the degradation of skin collagen, and improve the resiliency and youthful appearance of the skin.

To solve some of the above-mentioned problems, a novel composition containing a human decorin protein and a novel method of using same for effectively combacting aging of the skin are discovered in the present invention. Human decorin naturally exists in the skin to regulate skin collagen fiber formation, inhibit collagen fibrillogenesis, enhance collagen fibril stability, prevent collagen degradation, and normalize skin cells and elastin fibers. It is unexpectedly discovered in this invention that the sign of aging in the skin including loss of elasticity, fine lines, and wrinkles can be reduced, eliminated, or even reversed by using human decorin by topical application, leaving skin more resilient, smoother and youthful.

### BRIEF SUMMARY OF THE INVENTION

In brief, the discovery in the present invention of novel compositions containing human decorin and the methods of using same overcomes certain of the above-mentioned problems and shortcomings for effectively treating and preventing skin aging, repairing damaged skin from aging and restoring skin to a more resiliency and youthful appearance.

Accordingly, it is one object of the invention to provide a novel method for effectively treating and preventing skin aging, repairing damaged skin from aging and restoring skin to a more resiliency and youthful appearance through the topical administration of decorin with a suitable carrier or vehicle.

It is another object of the present invention to provide novel compositions which are useful in such methods.

It is another object of the present invention to provide novel formulations of decorin in a cosmetic or dermatological or pharmaceutical carrier or vehicle, which are effectively treating and preventing skin aging, repairing damaged skin from aging and restoring skin to a more resiliency, firmer, and younger appearance.

It is now discovered that decorin has unusual qualities as well as broader utilities which have not been disclosed in the prior art. Topical application of compositions containing decorin have been found to improve cosmetic as well as clinical signs of changes in skin associated with intrinsic aging or the damages caused by extrinsic factors such as UV radiations, radiations, chemicals, smoking, or weather.

It is another object of the present invention to provide a method for reducing, eliminating, or even reversing the sign of aging including loss of elasticity, fine lines, and wrinkles, by using decorin as an active agent by topical application.

A suitable carrier or vehicle will include the formulation of creams, gels, lotions, powders, sunscreens, cleansers, liquids, and various skin care preparations for the skin to repair the damage from aging, reduce further damage and restore skin to a more resiliency, firmer, and more youthful appearance.

It should also be understood that the particular methods and formulations illustrating the present invention are solely exemplary and consequently cannot limit the scope of the invention in any way.

### BRIEF DESCRIPTION OF THE DRAWING

FIG. 1 is a schematic representation of the construction of plasmid pZDGU9/DCN.

FIG. 2 is a schematic representation of the construction of plasmid pZDGU9.

FIG. 3 is a graph of the collagen fibrillogenesis inhibition assay, as set forth in Example 3.

FIG. 4 is a graph of the collagen protection assay, as set forth in Example 4.

FIG. 5 is a photo showing the results on the left-side facial skin after a 10-week application of the HDCP cream, as set forth in Example 6.

FIG. 6 is a photo showing the results on the right-side facial skin after a 10-week application of the control cream, as set forth in Example 6.

### DETAILED DESCRIPTION OF THE INVENTION

The present invention is directed to the use of decorin for the preparation of a cosmetic or pharmaceutical or dermatological composition for effectively treating and preventing aging of the skin and methods for effectively treating and preventing aging of the skin using such compositions.

1. Definition

In describing the present invention, the following terms are intended to be defined as indicated below.

“Recombinant” polypeptides refer to polypeptides produced by recombinant DNA techniques; i.e., produced from cells (microbial or mammalian) transformed by an exogenous DNA construct encoding the desired polypeptide. Polypeptide expressed in most bacterial cultures. e.g., E. coli, will be free of glycan. Polypeptide expressed in yeast may have a glycosylation pattern different from that expressed in mammalian cells.

“Native” proteins or polypeptides refer to proteins or polypeptides recovered from a source occurring in nature. Thus, the term “native decorin” would include naturally occurring decorin and fragments thereof.

A DNA “coding sequence” is a DNA sequence which is transcribed into mRNA and translated into a polypeptide in a host cell when placed under the control of appropriate regulatory sequences. The boundaries of the coding sequence are determined by a start codon at the 5′ N-terminus and a translation stop codon at the 3′ C.-terminus. A coding sequence can include prokaryotic sequences, cDNA from eukaryotic mRNA, genomic DNA sequences from eukaryotic DNA, and even synthetic DNA sequences. A transcription termination sequence will usually be located 3′ to the coding sequence.

“Nucleotide sequence” is a heteropolymer of deoxyribonucleotides (bases adenine, guanine, thymine, or cytosine). DNA sequences encoding decorin of this invention can be assembled from synthetic or cDNA-derived DNA fragments and short oligonucleotide linkers to provide a synthetic gene which is capable of being expressed in a recombinant expression vector. In discussing the structure of particular double-stranded DNA molecules, sequences may be described herein according to the normal convention of giving only the sequence in the 5′ to 3′ direction along the nontranscribed strand of DNA (i.e., the strand having the sequence homologous to the mRNA).

“Recombinant expression vector” is a replicable DNA construct used either to amplify or to express DNA encoding decorin of the present invention. An expression vector contains DNA control sequences and coding sequence. DNA control sequences include promoter sequences, ribosome binding sites, polyadenylation signals, transcription termination sequences, upstream regulatory domains and enhancers. Recombinant expression systems as defined herein will express decorin upon induction of the regulatory elements.

“Transformed host cells” refer to cells that have been transformed and transfected with exogenous DNA. Exogenous DNA may or may not be integrated (covalently linked) to chromosomal DNA making up the genome of the cell. In prokaryotes and yeast, for example, the exogenous DNA may be maintained on an episomal element, such as a plasmid or stably integrated into chromosomal DNA. With respect to eukaryotic cells, a stably transformed cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cell containing the exogenous DNA.

“PCR” means polymerase chain reaction which is based on a thermostable DNA polymerase from Thermus aquaticus. The PCR technique refers to a DNA amplification skill that mimics the natural DNA replication process in that the DNA molecules double after each thermal cycle, in a way similar to in vivo replication. The DNA polymerase mediates extension in a 5′ to 3′ direction. The “primer” refers to an oligonucleotide sequence that provides a 3′ end to which the DNA polymerase adds nucleotides complementary to a nucleotide sequence. The “template” refers to a nucleotide sequence to which the primers are annealed.

“Collagen Fibrillogenesis” refers to a process of formation of fibrils, in which soluble collagens become insoluble fibrils, lossing their tensile strength and elasticity.

“Cosmetic or dermatological or pharmaceutical acceptable carrier”, refers to a vehicle, for either cosmetic, dermatological or pharmaceutical use, which delivers the active components to their site of action and will not cause harm to human or animal recipient.

“Skin Penetration Enhancer” refers to a vehicle, for either cosmetic, dermatological or pharmaceutical use, which help to transport the active compoments through the normal intact skin.

2. Decorin

The term decorin refers to proteins having amino acid sequences which are substantially similar to the native human decorin amino acid sequences and which are biologically active in that they are capable of binding to decorin receptors, transducing a biological signal initiated by binding decorin receptors, or cross-reacting with anti-decorin antibodies raised against decorin. DNA sequences encoding decorin are disclosed, for example, in Krusius and Ruoslahti, Proc. Natl. Acad. Sci. USA 83:7683, 1986 and Day et al., Biochem. J. 248:801, 1987.

Decorin modulates collagen fiber formation, inhibits fibrillogenesis and maintains collagen solubility and elasticity, and normalizes the morphology of elastin fibers and skin cells. Decorin is discovered in this invention to have an important biological function in retarding aging of the skin. Decorin prevents degradation of collagen and resists degradation by MMP. Decorin used as an active agent by topical application can tone down big wrinkles and cause the disappearance of small wrinkles and fine lines, restoring skin a more resiliency and smoother and youthful-look.

3. Proteins and Analogs

The present invention includes a decorin-containing composition for treating and preventing aging of the skin. Derivatives and analogs of decorin of the present invention may also be obtained by modifying the primary amino acid structure with other chemical moieties, by mutations of decorin, by linking particular functional groups to amino acid side chains or at the N- or C-termini, or by conjugating decorin with other proteins or polypeptides. Bioequivalent analogs of decorin may also be constructed by making various substitutions of residues or sequences.

4. Construction of cDNA Sequences Encoding Decorin

A DNA sequence encoding decorin is constructed using recombinant DNA techniques into an appropriate expression vector with the reading frames of the DNA sequences in phase to permit mRNA translation of the sequences into a biologically active decorin protein.

5. Expression of Decorin

There are several ways to express decorin in vitro, including in E. coli, insect cells, yeast, mammalian cells or other expression systems.

The prokaryotic system, E. coli, is not able to do post-translational modification, such as glycosylation. But this is probably not a problem for decorin since decorin is not heavily glycosylated (Krusius and Ruosslahti, Proc. Natl. Acad. Sci. USA 83:7683, 1986). It has reported that E. coli-derived human decorin maintains its binding functions to collagen and fibronectin (Hering et al., Anal. Biochem. 240:98, 1996). Decorin may also be expressed in insect cells such as Spodoptera frugiperda 21 or in yeast hosts such as Saccharomyces cerevisiae and Pichia pastoris. It usually gives the ability to do various post-translational modifications. Mammalian cell lines, such as the COS-7, L cells, C127, 3T3, Chinese hamster ovary (CHO), Hela and BHK, can be employed to express decorin as well. Decorin produced in mammalian cells are normally soluble and glycosylated and have an authentic N-terminal.

Depending on the expression system and host selected, a homogeneous decorin can be obtained by some of the purification steps, in various combinations, of the conventional chromatographys of protein purification, which include immunoaffinity chromatography, reverse phase chromatography, cation exchange chromatography, anion exchange chromatography, hydrophobic interaction, hydroxyapatite chromatography, gel filtration chromatography and high performace liquid chromatography (HPLC). If the expression system secretes decorin into growth media, the protein can be purified directly from the media. If decorin is not secreted, it is isolated from cell lysates. Cell disruption can be done by any conventional method, including freeze-thaw cycling, sonication, mechanical disruption, or use of cell lysing agents.

6. Cosmetic or Dermatological or Pharmaceutical Composition Containing Decorin

Although it has been known that human decorin inhibits in vitro collagen fibrillogenesis, there is an unexpected role discovered in the present invention of decorin in protecting collagen from degradation by collagenase. It is also discovered that decorin itself is able to resist degradation by collagenase and other proteases, such as caseinase and clostripain. Futhermore, decorin-containing cosmetic or dermatological compositions discovered in the invention has an unexpected and surprising effect on the aging sign of the skin when it is topically administrated. Decorin used as an active agent by topical application can tone down big wrinkles and reduce or eliminate small wrinkles and fine lines after a 10-week administration, restoring skin a more resiliency and smoother and youthful look.

Decorin is present in the compositions of the invention in an amount of about 0.5 to about 5,000 mu.g/ml, preferably about 5 to about 500, and most preferably about 40 mu.g/ml.

The decorin-containing compositions of the present invention can be combined with additional active agents. These agents include, but are not limited to, decorin-synthesis enhencers, collagen-synthesis enhencers, matrix metalloproteinases (MMP) inhibitors, antioxidants, collagen modulators, anti-wrinkle or anti-aging agents, antibiotics, depigmenting agents, analgesics, antimicrobials, anti-inflammatory agents, moisturers, skin lightening agents, corticosteroids, or sun-block agents.

The decorin-containing compositions can be combined with a cosmetically or pharmaceutically or dermatologically acceptable carrier. The carriers include, but are not limited to, water, mineral oil, ethylene glycol, propylene glycol, lanolin, glyceryl stearate, sorbitan stearate, isopropyl myristate, isopropyl palmitate, acetone, glycerol, phosphatidylcholine, sodium cholate, or ethanol.

The decorin-containing compositions can be combined with a skin penentration enhancer. The enhancers, helping to transport the active components through the normal intact skin, include, but are not limited to, liposomes, mixed lipid micelles, ethosomes, transfersomes, niosomes, ethanol, amides, ethers, glycols, hydrocarbon oils, sodium lauryl sulfate, oleic acid, hydroalcoholic solution, and soya phosphatidylcholine or their combinations. Other skin penetration enhancement includes different pH values, co-solvents, surfactants, cyclodextrins, and iontophoresis.

A suitable carrier or vehicle or enhancer will include the formulation of gels, creams, lotions, solutions, colloidal dispersions, emulsions (oil-in-water or water-in-oil), foams, sprays, suspensions, sunscreens, liquid and various skin care preparations for topical application to the skin. The decorin-containing compositions can be prepared in any formula for topical application to the skin.

The formulation mentioned above can also be combined with other ingredients, depending on the intended use of the formulation. These ingredients include, but are not limited to, preservatives, vitamins, polymers, fragrances, water- or oil-soluble filmformers, or flavoring agents.

The present invention also provides methods of treating the skin of a human to combact skin aging, comprising applying to the skin a cosmetic or dermatological or pharmaceutical compostion containing an effective amount of decorin. The compositions of the invention can be applied to the skin on an as-needed basis, for example, they can be applied to the skin in the morning and/or in the evening, and/or during the day. It is suggested that topical application be once a month to about 7 or 8 times daily, preferably from about 7 times a week to about 4 times a day, most preferably about twice a day. However, a preferred method of obtaining the benefits of the composition is a continuing topical application of a composition containing an effective amount of decorin, to prevent the loses of collagen solubility and elasticity, to prevent the degradation of collagen, to repair the damage of the skin associated with intrinsic or extrinsic aging, to normalize the morphology of elastin fibers and skin cells, and to restore the skin to be resiliency, firm, and youthful. It is also suggested to maintain the effects of the composition on the skin that the period of topical application may be over the lifetime of the user, preferably for a period from 4 weeks to twenty years, more preferably from about 6 months to about five years, resulting in the treatment or prevention of the signs of aging in the skin.

The decorin-containing compositions in the present invention can also be a product of sunscreen, or makeup products, or others, which include, but are not limited to, perfumes, humectants, or fragrants.

The following examples are offered to further illustrate the invention and are not intended to be limitative thereof:

### EXAMPLE 1

Synthesis of cDNAs Encoding Human Decorin Core Protein (HDCP)

The double-stranded cDNA of HDCP was synthesized from human skin fibroblast cDNA library purchased from Clontech (Palo Alto, Calif.) by using Taq DNA polymerase and a set of upstream and downstream oligonucleotide primers for HDCP. The primers are listed in Table 1. The 5′ primer (Dcn-A) contained a NcoI site and the coding sequence for the first 7 amino acids from the HDCP. The 3′ primer (Dcn-B) contained a EcoRI site and coding sequence for the last 6 amino acids from the HDCP. The PCR buffer contained 50 mM KCl, 10 mM Tris-HCl (pH 9.0), 1.5 mM MgCl.sub.2, 0.01% gelatin, 0.05 mmol each of dNTP, 1.0 mu.mol of each primers, 10 mu.l reverse transcription reaction mixture, and 2 units of Taq DNA polymerase in a total of 50 mu.l volume. The PCR condition was 94 degree C. for 30 seconds, 55 degree C. for 30 seconds, and 72 degree C. for 30 seconds for 25 cycles in the MJ Research model PTC-1152 thermocycler (MJ Research, Watertown, Mass.).

TABLE 1
Primers used in PCR to synthesize human decorin core protein

| Designation | Primer Sequence | Primer Length |
|---|---|---|
| Dcn-A | 5′ GCCATGGATGAGGCTTCTGGGATGGGCC 3′ (SEQ ID NO: 1) | 28 |
| Dcn-B | 5′ AGAATTCTATTACTTATAGTTTCCGAGTTG 3′ (SEQ ID NO: 2) | 30 |

The PCR amplified DNA fragments were gel-purified and cloned into pGEM-T vectors (Promaga, Madison, Wis.). The PCR cDNA fragments with 3′-A overhangs can be directly ligated into pGEM-T cloning vectors without any digestion of restriction endonuclease. After ligation, the DNA was then transformed into competent E. coli DH5.alpha.cells. Designing the restriction endonuclease sites in the primers is for subcloning the cDNA fragments into expression vectors.

The plasmid isolated from one of the colonies was confirmed to contain the right size of the insert (approximate 1.0 kb) by the analyses of restriction endonucleases and to comprise a DNA sequence of HDCP by DNA sequence in both directions by the chain termination method (Sanger et al., Pro. Natl. Acad. Sci. 74:5463, 1977). The primers for the DNA sequencing are listed in Table 2.

TABLE 2
Primers used for sequencing

| Designation | Primer Sequence | Primer Length |
|---|---|---|
| Sp6 | 5′ CTATTTAGGTGACACTATAG (SEQ ID NO: 3) | 20 |
| T7 | 5′ TAATACGACTCACTATAGGG (SEQ ID NO: 4) | 20 |

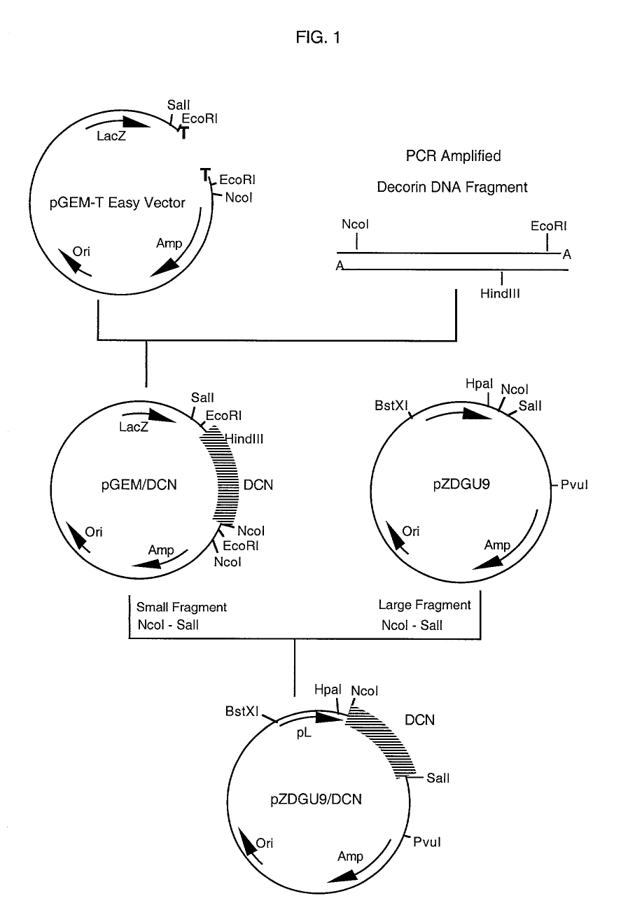

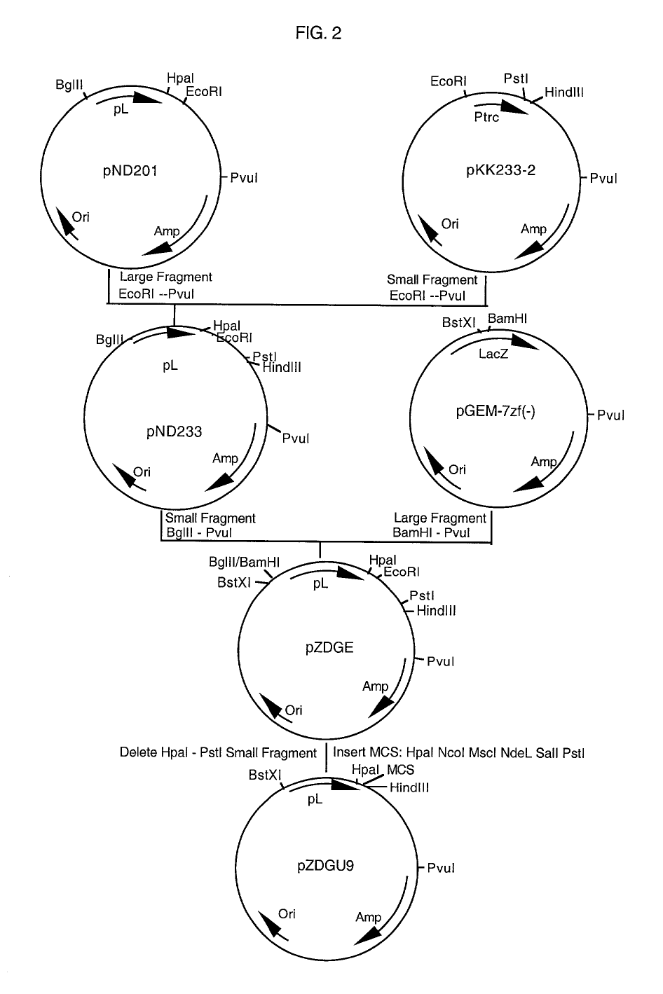

The plasmid containing the DNA insert encoding HDCP is designated as pGEM/Dcn (FIG. 1). Plasmid pGEM/Dcn was digested with restriction endonucleases NcoI and SalI to release the DNA insert encoding the HDCP. The DNA fragments were gel purified and then ligated to the prokaryotic PL (phage lamda left promoter) expression vectors pZDGU9 (FIG. 2) through the NcoI and SalI sites. After ligation, the pZDGU9/Dcn DNA was transformed into competent E. coli strain N4830-1 purchased from Pharmacia (Peapack, N.J.). N4830-1 contains the temperature-sensitive cI857 mutation.

The competent cells of N4830-1 were prepared by the CaCl.sub.2 method (Mandel and Higa, J. Mol. Biol. 53:159, 1970). Briefly, 50 ml of LB medium without antibiotics was inoculated with a single E. coli N4830-1 colony and the culture grown overnight at 30 degree C. with shaking at 250 rpm. The overnight culture was diluted 1:50 with LB medium without any antibiotic and continued the cultivation at 30 degree C. with 250 rpm until an OD.sub.590 reaches 0.3-0.5. The culture was then placed on ice for 10 minutes and centrifuged 10 minutes at 3000 rpm at 4 degree C. The supernatant was discarded. The cell pellet was resuspended gently in 40% of the starting volume with the ice-cold 0.1 M CaCl.sub.2 solution. The cell suspension was kept on ice for 30 minutes and then spinned down at 3000 rpm for 10 minutes at 4 degree C. The pellet was resuspended again in 2% of the starting volume with the ice-cold 0.1 M CaCl.sub.2 solution, transferred into a sterile polypropylene tube, and then chilled on ice overnight at 4 degree C. Cold sterile 80% glycerol in distilled water was added into the cell suspension to a final concentration of 20% and mixed gently. The competent cells, at a density of approximately 1 times 10.sup.9/ml were stored in a 40 microliter aliquot at −70 degree. C. For transformation, 35 microliter of N4830-1 competent cells were thawed on ice and transferred into an eppendorf tube containing approximately 5 ng pZDGU9/Dcn DNA. The mixture was left on ice for 30 minutes and mixed by swirling gently. The cells were heat-shocked at 42 degree C. for exactly 45 seconds in a circulating water bath that has been preheated at 42 degree C. The cells were rapidly returned to an ice bath and allowed to chill for 2 minutes. Ten volumes of SOC medium were added to the tube. The cells were incubated at 28 degree C. for 60 minutes with shaking at 250 rpm to allow the bacteria to recover and to express the antibiotic resistant marker encoded by the plasmid. Transformed competent cells were transferred onto 90-mm agar plates containing the antibiotic and gently spread over the surface of the agar plate using a sterile bent glass rod. The plates were left at room temperature until the liquid has been absorbed. The plates were then inverted and incubated at 28 degree C. overnight. The plasmid isolated from one of the colonies was confirmed by the analyses of restriction endonucleases and DNA sequence in both directions to comprise decorin cDNA containing a mutation of an adenine to a thymine at nucleotide position 2 (SEQ ID NO:5). Its corresponding amino acid sequence in SEQ ID NO:6 shows that the asparagine was replaced by a valine at amino acid position 1. The plasmid is designated as pZDGU9/Dcn (FIG. 1). The pZDGU9 expression vector constructed according to FIG. 2 is derived from pND201 vector (ATCC# 37831), which contains tandem-arranged bacteriophage lambda promoters, P.sub.R and P.sub.L, and the lambda c1857 gene (Elvin, et al., Gene 87:123, 1990).

Plasmid pZDGU9/Dcn is deposited with the American Type Culture Collection (ATCC) as a patent deposit at 10801 University Blvd., Manassas, Va. 20110: Accession number: PTA-2223; Deposit date Jul. 12, 2000 (E. coli N4830-1/pZDGU9/Dcn as the host vector system). Plasmid pZDOU9/Dcn is a recombinant expression vector comprising a DNA sequence (SEQ ID NO:5) that encodes human decorin core protein (SEQ ID NO:6).

### EXAMPLE 2

Expression and Purification of Human Decorin Core Protein (HDCP)

The E. coli N4830-1 cells containing the expression vector pZDGU9/Dcn were grown overnight in LB broth containing 100.mu.g/ml ampicillin at 28 degree C., with rotary shaking at 225 rpm in a New Brunswick Incubator Shaker, Model King Size G24 (Edison, N.J.). The overnight culture was diluted 1:10 with IX LB culture medium. The N4830-1 cells were grown at 28 degree C. until the OD.sub.680 of the culture reached 3.0 at which time the temperature was raised to 42 degree C. and ZnSO.sub.4 solution (100 mM) was added to the culture to a final concentration of 0.5 mM. The cultivation was continued for another 6 hours in that matter. The cells were harvested by centrifugation and the bacterial pellets were stored at −70 degree C. until further purification.

For purification of human decorin core protein, the frozen E. coli cell pellets were suspended in 6 volumes of lysis buffer (50 mM Tris HCl, pH 8.0, 1 mM EDTA, 1 mM DTT, 1 mM phenylmethanesulfonyl fluoride, 2 mg/ml lysozyme) and disrupted by sonication with Branson Digital Sonifier 450 (Danbury, Conn.). The settings of sonication were 50 ml aliquotes with a ⅜ inch probe, 80W, pulse on 10 seconds and off 30 seconds, on ice for 6 minutes. The insoluble inclusion bodies (IB) isolated from the cell lysate by centrifugation were washed 3 times with 1% Triton in 50 mM Tris buffer, pH 8.0 to reach approximate 70% purity. The inclusion bodies were then solubilized in 8 M urea in 100 mM Tris-acetate/NaOH, pH 9.5, 25 mM EDTA, 5 mM DTT. Extracted HDCP were subjected to refolding reaction at a redox buffer (50 mM Tris HCl, pH 9.0, 5 mM EDTA, 0.2 mM oxidized glutathione, 0.4 mM reduced glutathione and 0.5 mM ZnSO.sub.4.). The renatured HDCP solution was further purified to homogeneity in a series of column chromatographic purifications.

Briefly, the renatured HDCP solution was loaded onto a high S column equilibrated with 25 mM sodium acetate buffer, pH 5.5. The column was washed with the equilibrated buffer until the absorbance of the eluate was zero or nearly zero, and then eluted with acetate buffer (25 mM sodium acetate, pH 5.5, 300 mM NaCl). The protein pool adjusted to pH 6.8 with 1.0 M sodium phosphate buffer, pH 6.8 was added with solid ammonium sulphate to a final concentration of 2.0 M and loaded onto a hydrophobic interaction column equilibrated with 2.4 M ammonium sulphate, 10 mM sodium phosphate, pH 6.8. After washed with 25 column volume of equilibrated buffer, the column was liberated with 10 mM sodium phosphate, pH 6.8. The protein pool was then loaded onto a hydroxyapatite column equilibrated with 10 mM sodium phosphate, pH 6.8. After washed with 25 column volume of the equilibrated buffer, HDCP was eluted with 300 mM sodium phosphate, pH 6.8. The eluate from the hydroxyapatite column was concentrated with an Amicon concentrator Stirred cell 2000 and then applied to a gel filtration column (Sephacryl S-200 HR) equilibrated with 1×PBS buffer, pH 7.3. The fusion protein peak was then recovered. All the purification steps were carried out at 4 degree C.

The HDCP samples were analyzed under standard reducing conditions in 8% SDS polyacrylamide gel electrophoresis (SDS-PAGE) (Laemmli, Nature 277:680, 1970). The protein bands were visualized by Coomassie blue staining. The apparent molecular weight of HDCP is about 40 kd and its PI is about 8.7. The concentration of HDCP was determined with the BioRad Protein Assay. This assay uses the dye Coomassie brilliant blue and measures the protein/dye complex at 595 nm. The standard used is bovine serum albumin.

### EXAMPLE 3

Collagen Fibrilogenesis Inhibition Assay

Human collagen Type I (acid soluble) was purchased from Sigma (St. Louis, Mo.) and dissolved in 10 mM acetic acid, pH 3.4, at a concentration of 0.4 mg/ml, aliquoted and stored at −70 degree C. Purified human decorin core protein (HDCP) was suspended in 1×PBS (phosphate-buffered saline) at a concentration of 100 mcg/ml and stored at −70 degree C. All buffer solutions in this assay were degassed and kept at 4 degree C. The assay was carried out according to the method described by Vogel et al (Biochem. J. 223:587, 1984).

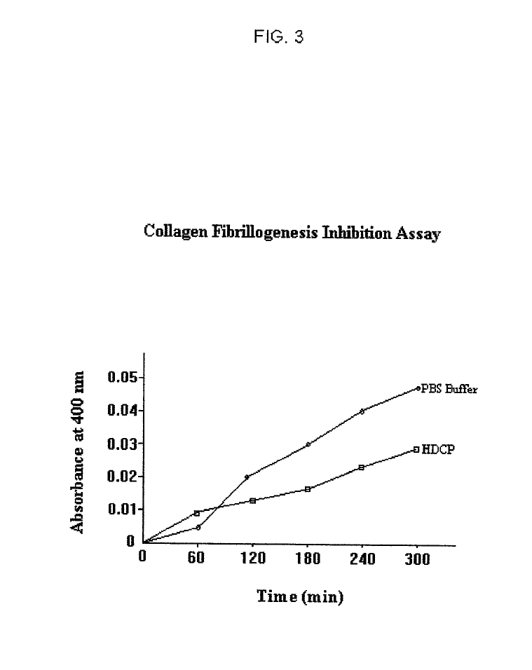

20 microliters (80 mcg) of human collagen Type I solution of 0.4 mg/ml were diluted with 230 microliters of 10 mM acetic acid, pH 3.4 and 250 microliters of 1×PBS. 50 microliters (5 mcg) of HDCP solution of 100 mcg/ml were diluted with 200 microliters of 1×PBS. These two solutions above were combined and then added with 250 microliters 2×PBS to a final volume of 1.0 ml and mixed well by pipetting up and down for 4-5 times. Approximately 0.8 ml of the mixed solution above was quickly drawn into a thermocuvette whose temperature was pre-set for 37 degree C. in Gilford Stasar III Spectrophotometer (Ciba Coming Diagnostic Corp, Oberlin, Ohio). Settings for Stasar III were vaccum regulator at 10 inches Hg, sample timer control at 5, ABS-Con-ACC control at ABS and wavelength at 400 nm. Fibrillogenesis was monitored by the absorbance at 400 nm at 5-minute intervals up to 4 hours. Control experiments were carried out by substitution of HDCP with 1×PBS. The data represented in FIG. 3 indicates that recombinant HDCP can inhibit fibril formation at 37 degree C.

### EXAMPLE 4

Collagen Protection Assay

20 microliters (80 mcg) of human collagen Type I solution of 0.4 mg/ml in 10 mM acetic acid, pH 3.4, were mixed well with 80 microliters (8 mcg) of HDCP solution of 100 mcg/ml in 1×PBS, pH 7.3, and with 100 microliter 2×buffer containing 50 mM TES [N-tris(hydroxymethy)methyl-2 aminoethanesulfonic acid], 0.36 mM CaCl.sub.2, pH 7.4. After incubation for 10 minutes at 25 degree C., the mixture was added with 20 ng crude human collagenase Type I (Sigma) solution of 10 mcg/ml and mixed well. Before the reaction solution was transferred to a 37 degree C. water bath, 20 microliters of the solution were taken out as a sample of zero time digestion. Then a 20-microliter sample was taken out at each 30-minutes intervals so that the time frame in this assay was 0, 30, 60 and 90 minutes, respectively. All samples were mixed with 5 microliter of 6×SDS-PAGE (SDS-polyacrylamide gel electrophresis) loading buffer right after being taken out from the reaction and kept in −20 degree C. freezer for further analysis. Control experiment was also carried out. All conditions for the control were the same as above except using PBS to substitute human decorin core protein. Samples were boiled for five minutes and dissolved in a 8% SDS-PAGE under reducing conditions. The protein bands were visualized with Coomassie Blue R-25 and analyzed with Bio-Rad Calibrated Imaging Densitometer System: GS-710 (Bio-Rad, Hercules, Calif.). The data [data units: optical density/mm²] are listed in the Table 3 below.

TABLE 3
Collagen Protection Assay [Time (minute) vs OD (mm²)]

| Incubation Time (min.) | 0 | 30 | 60 | 90 |
|---|---|---|---|---|
| C + PBS + Z | 9.5302 | 4.6065 | 1.7491 | 1.5394 |
| C + D + Z | 10.3549 | 9.3714 | 5.8902 | 1.6717 |
| D | 3.6015 | 2.5645 | 2.1376 | 2.3488 |

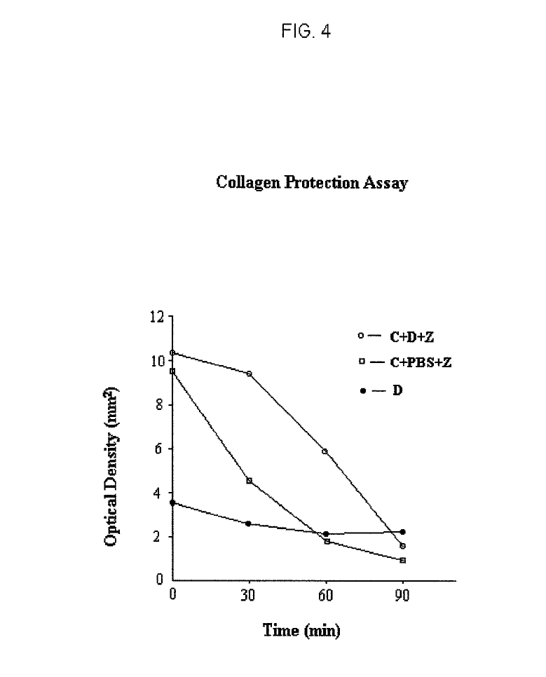

The abbreviations: C (human collagen type I), Z (human collagenase type 1), D (human decorin core protein), PBS (phosphate-buffered saline), OD (optical density). The data indicates that human decorin core protein has a significant role in the protection of collagen from degradation by collagenase in the 30- or 60-minutes reaction (FIG. 4). The degradation rate of collagen in the reaction with HDCP is 9.49% in the 30-minutes reaction and 37.15% in 60-minutes reaction. On the other hand, the rate increases up to 51.66% in the 30- and 62.03% in the 60-reactions when the reaction is without HDCP (Table 4). It appears that collagen loses its protection from HDCP in the 90-minutes reaction. HDCP can stand a long time incubation with collagenase with a little degradation and also resists other protease, such as caseinase, clostripain, which coexist in the crude collagenase I. Based on the Certificate of Analysis carried out by Sigma (St. Louis, Mo.), each milligram solid crude collegenase type I (lot# 060k8614) used in this experiment contains 637 collagen digestion units, 0.54 FALGPA hydrolysis units, 118 caseinase units and 0.8 clostripain units.

TABLE 4
Collagen Type I Degradation Rate (%)

| Incubation Time (Min.) | 0 | 30 | 60 | 90 |
|---|---|---|---|---|
| C + PBS + Z | 0 | 51.66% | 62.03% | 11.98 |
| C + D + Z | 0 | 9.49% | 37.15% | 71.62% |
| D | 0 | 28.79% | 16.65% | 0% |

### EXAMPLE 5

Preparation of a HDCP-Containing Lipidvesicles

A modified method was used to prepare HDCP-containing lipidvesicles, according to Cevc et al., Biochim. Biophys. Acta 1368:201, 1998. In brief, 4.3 gram of hydrogenated soybean lecithin and 0.7 gram of sodium cholate purchased from ICN Biomedicals (Costa Mesa, Calif.) were dissolved in 4.2 ml of dehydrated ethanol for 30 minutes. 30.8 ml of 50 mM triethanolamine-HCl, pH 6.8, was added to the suspension and sonicated by using a Branson Digital Sonifier 450 (Danbury, CT) with a ⅜ inch probe, 80W, pulse on 10 seconds and off 30 seconds for 30 minutes on ice. The suspension was then frozen, thawed, and sonicated again for 5 times to create a lipidvesicle suspension. The 10 mg HDCP suspended in 1×PBS at a concentration of 1.0 mg/ml was incorporated in such carriers by mixing them together by stirring and incubating them for 48 hours at 4 degree C. The final concentration for lipids is 10% and for HDCP 0.02%. The final lipid/HDC ratio is approximately 1 times 10.sup.6 (1.0×10⁶) mol/mol. The control experiment was carried out by adding 10 ml PBS without HDCP. The resulting suspension was filtered through with a 0.2 micrometer filter and stored at 4 degree C. for further preparation. The mean size of the HDCP-containing vesicle is approximatly 100 nm.

### EXAMPLE 6

Preparation of a HDCP-Containing Face Cream

| Phase A: | |
|---|---|
| HDCP-containing vesicle | 20% |
| Phase B: | |
| Mineral oil | 10% |
| Hydrogenated lanolin | 8% |
| Glyceryl stearate | 3% |
| Sorbitan stearate | 1% |
| Phase C: | |
| Glycerol | 3% |
| Preservative | 0.4% |
| Demineralized water | 52.6% |

The phase B and C were heated separately to 80 degree C. The phase B was then poured into the phase C with stirring to create an emulsion. The emulsion was stirred continuously until its temperature was cooled down to 30 degree C. Meanwhile, the phase A was thawed out, warmed up to room temperature and then poured into the emulsion. The mixture was mixed well by stirring until it cooled down to room temperature. A white HDCP cream was obtained. The final concentration of HDCP is 4 mg per 100 ml cream (40.mu.g/ml or 0.004%) and the final buffer concentration is 10 mM triethanolamine-HCl, pH 6.8.

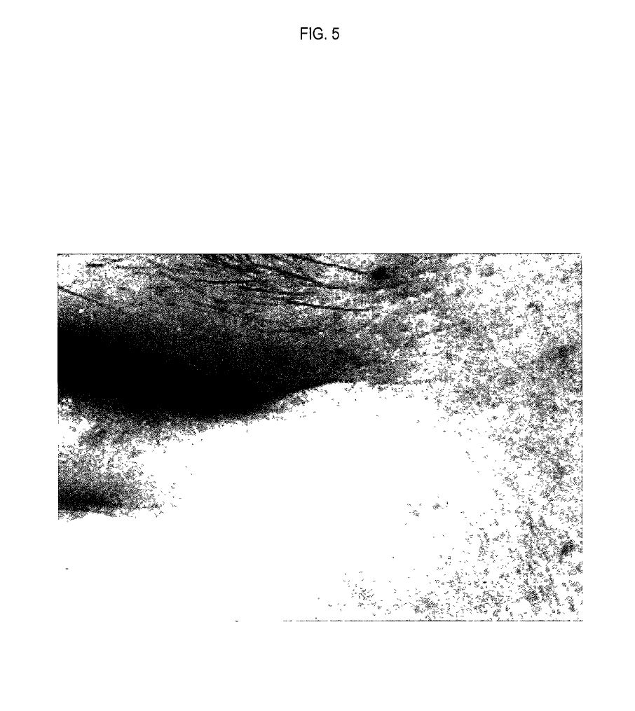

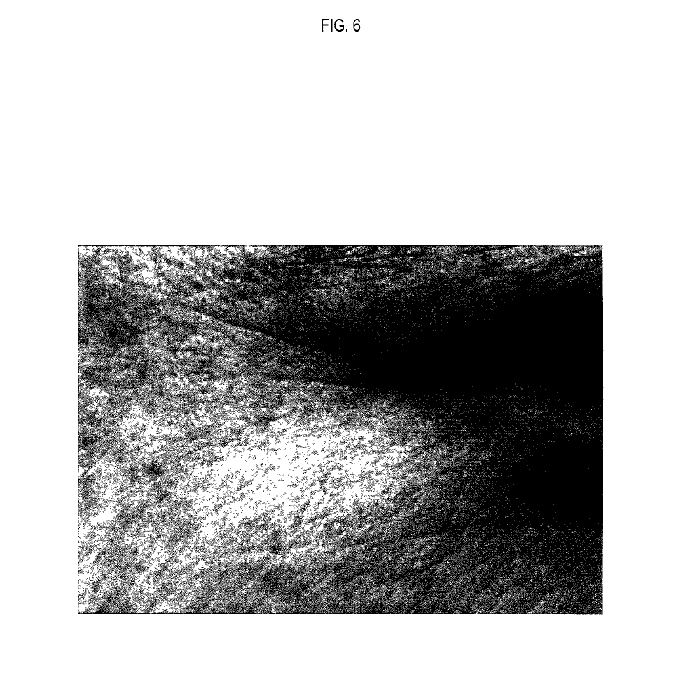

The HDCP cream was applied to one side of the facial skin area about 4 cm.sup.2 between the temple, the outer canthus, and the upper cheek. A control study was also carried out at the same time on the other side of the facial skin by using a control cream. The control cream has the same ingredients as the HDCP cream except it does not contain HDCP. 24 volunteers ranging from 21 to 50 years old were involved. Each volunteer was given two jars of color-labeled creams, the HDCP and the control. To prevent prejudice, volunteers were not told which cream contained HDCP or which was the control until the end of the study. The volunteers were told to use only one cream on one side of the facial skin 2 times a day (morning and night) for 10 weeks. The results indicated that, in the age group 21-30, the facial skin on the side using the HDCP cream was smoother than the control side and the occurence of a fine line was retarded. In the age group 31-40, fine lines disappeared and the skin was smoother. In the age group 41-50, fine lines and small wrinkles were significantly reduced or diminished. Large wrinkles were dramatically toned down. The skin appeared smoother and youthful. FIGS. 5 and 6 are facial photos magnified approximately by 3.5 fold, illustrating examples of a comparison between the left-side facial skin and the right-side facial skin of a person in the age group 41-50 after a 10-week application of the HDCP cream and the control cream. FIG. 5 shows a remarkable wrinkle reduction and a considerable restoration of a youthful-look on the left-side facial skin after a 10-week application of the HDCP cream. FIG. 6 shows little or no change in wrinkle reduction or the improvement of a youthful appearance on the right-side after a 10-week application of the control cream which does not contain HDCP.

I claim:

1. A cosmetic or pharmaceutical or dermatological compostion containing an effective amount of decorin dissolved or dispersed for topical administration in a cosmetically or dermatologically or pharmaceutically acceptable vehicle.

2. The composition of claim 1, wherein said decorin is naturally derived or produced by recombinant means.

3. The composition of claim 2, wherein said decorin comprises the human mature core protein set forth as amino acids 1-329 of Sequence ID 6.

4. The composition of claim 3, wherein said human decorin core protein is present in an amount between about 0.5.mu.g/ml and about 5,000.mu.g/ml.

5. The composition of claim 4, wherein said decorin is present in an amount between about 5.mu.g/ml to about 500.mu.g/ml.

6. A method of treating the skin of a human to combat aging, comprising applying to the skin a cosmetic or dermatological or pharmaceutical compostion containing an effective amount of decorin dissolved or dispersed in a cosmetically or pharmaceutically acceptable vehicle.

7. The method of claim 6 wherein the composition comprises decorin naturally derived or produced by recombinant means.

8. The method of claim 7 wherein the composition comprises the human decorin core protein set forth as amino acids 1-329 of Sequence ID 6.

9. The method of claim 8 wherein the composition comprises decorin in an amount from about 0.5.mu.g/ml to about 5,000.mu.g/ml.

10. The method of claim 9 wherein the composition comprises decorin in an amount from about 5.mu.g/ml to about 500 mu.g/ml.
